SEQUENCE LISTING (10 sequences)

SEQ ID NO: 1 - protein, 5 aa, Artificial Sequence
  Synthetic Construct
AYSVN

SEQ ID NO: 2 - protein, 16 aa, Artificial Sequence
  Synthetic Construct
MIWGDGKIVYNSALKS

SEQ ID NO: 3 - protein, 10 aa, Artificial Sequence
  Synthetic Construct
DGYYPYAMDN

SEQ ID NO: 4 - protein, 15 aa, Artificial Sequence
  Synthetic Construct
RASKSVDSYGNSFMH

SEQ ID NO: 5 - protein, 7 aa, Artificial Sequence
  Synthetic Construct
LASNLES

SEQ ID NO: 6 - protein, 9 aa, Artificial Sequence
  Synthetic Construct
QQNNEDPRT

SEQ ID NO: 7 - protein, 117 aa, Artificial Sequence
  Synthetic Construct
VTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 8 - protein, 111 aa, Artificial Sequence [sequence omitted]

SEQ ID NO: 9 - protein, 445 aa, Artificial Sequence
  Synthetic Construct
  MISC_FEATURE (1)..(1): Xaa at position 1 is Q, pE, or absent
  MISC_FEATURE (444)..(444): Xaa at position 444 is G or absent
  MISC_FEATURE (445)..(445): Xaa at position 445 is K or absent
XVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLXX

SEQ ID NO: 10 - protein, 218 aa, Artificial Sequence
  Synthetic Construct
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0289882 A1
(43) Pub. Date: Sep. 18, 2025

(54) IL-13 ANTIBODIES FOR THE TREATMENT OF ATOPIC DERMATITIS

(71) Applicant: DERMIRA, INC., Indianapolis, IN (US)

(72) Inventors: Helena AGELL GIMENO, Girona (ES); Clara ARMENGOL TUBAU, Barcelona (ES); Maria Esther GARCIA GIL, Barcelona (ES); Silvia MAESO NAVAL, Barcelona (ES)

(21) Appl. No.: 18/579,331

(22) PCT Filed: Jun. 30, 2022
(86) PCT No.: PCT/US2022/035663

§ 371 Date: Jan. 13, 2024

(30) Foreign Application Priority Data
Jul. 16, 2021 (EP) 21382645.6
Feb. 7, 2022 (EP) 22382098.6

(51) Int. Cl. C07K 16/24; A61K 31/573; A61K 31/58; A61K 39/00

(52) U.S. Cl. CPC C07K 16/244; A61K 31/573; A61K 31/58; A61K 2039/54; A61K 2039/545; C07K 2317/565; C07K 2317/76

(57) ABSTRACT

Provided herein are methods, uses and pharmaceutical compositions of antibodies that bind human IL-13 (“anti-IL-13 antibodies”) for treating atopic dermatitis or reducing pruritis associated with atopic dermatitis. Also provided herein are doses and dosing regimens for the methods and uses of anti-IL-13 antibodies for treating atopic dermatitis or reducing pruritis associated with atopic dermatitis.

### SEQUENCE LISTING

The present application is being filed along with a Sequence Listing. The Sequence Listing is provided as a file titled “X23042_US_000 Sequence Listing ST25_ST25” created May 20, 2025, and is 12 kilobytes in size. The Sequence Listing information in the ASCII text file format is incorporated herein by reference in its entirety.

### FIELD

The present invention relates to methods, uses and pharmaceutical compositions of antibodies that bind human IL-13 (“anti-IL-13 antibodies”) for treating atopic dermatitis or reducing pruritis associated with atopic dermatitis. The present invention also relates to doses and dosing regimens for the methods and uses of anti-IL-13 antibodies for treating atopic dermatitis or reducing pruritis associated with atopic dermatitis.

### BACKGROUND

Atopic dermatitis (AD) is a chronic relapsing and remitting inflammatory skin disorder affecting all age groups. Clinically, AD is characterized by xerosis, erythematous crusting rash, lichenification, an impaired skin barrier, and intense pruritus (Bieber T., N Engl J Med 2008;358:1483-94). Patients with AD have a high disease burden, and their quality of life is significantly impacted. In one study, AD was shown to have a greater negative effect on patient mental health than diabetes and hypertension (Zuberbier T, et al., J Allergy Clin Immunol 2006;118:226-32). Patients with moderate to severe AD have a higher prevalence of social dysfunction and sleep impairment, which is directly related to severity of disease (Williams H, et al., J Allergy Clin Immunol 2008;121:947-54.e15). Depression, anxiety, and social dysfunction not only affect patients with AD but also their caregivers (Zuberbier T, et al., J Allergy Clin Immunol 2006;118:226-32).

Interleukin (IL)-13 is a key mediator of T-helper type 2 (Th2) inflammation and signals through a heterodimeric receptor IL-4Rx/IL-13Ra1. Several lines of evidence suggest that IL-13 is a key pathogenetic component in AD. Increased expression of IL-13 has consistently been

reported in AD skin (Hamid Q, et al., J Allergy Clin Immunol 98:225-31 [1996]; Jeong C W, et al.,

Clin Exp Allergy 33:1717-24 [2003]; Tazawa T, et al., Arch Dermatol Res 295:459-64 [2004];

Neis MM, et al., J Allergy Clin Immunol 118:930-7 [2006]; Suarez-Fariñas M, et al., J Allergy Clin

Immunol 132:361-70 [2013]; Choy D F, et al., J Allergy Clin Immunol. 130:1335-43 [2012]) and some reports suggest a relationship between IL-13 expression and the severity of disease (La Grutta S, et al., Allergy 60:391-5 [2005]). Increased IL-13 has also been reported in the serum of AD patients (Novak N, et al., J Invest Dermatol 2002;119:870-5; WO2016149276), and several studies have reported an increase in IL-13-expressing T cells in the blood of AD patients (Akdis M, et al., J Immunol 1997;159:4611-9; Aleksza M, et al., Br J Dermatol 2002;147:1135-41; La Grutta S, et al., Allergy 2005;60:391-5).

The therapeutic approaches to AD primarily include trigger avoidance, skin hydration with bathing and use of emollients and anti-inflammatory therapies such as topical corticosteroids (TCS). In many patients, treatment with TCS provides some measure of symptomatic relief but does not adequately control their disease. In addition, TCS use is associated with many comorbidities and limitations including high patient burden. Long-term application of TCS is not recommended because of the risk of skin atrophy, dyspigmentation, acneiform eruptions, and risks associated with systemic absorption (e.g., hypothalamic pituitary axis effects, Cushing's disease).

For patients who have persistent moderate to severe AD and do not respond adequately to TCS, there are a number of step-up therapeutic options (Ring J, et al., J Eur Acad Dermatol Venereol 2012;26:1176-93; Schneider L, et. al., J Allergy Clin Immunol 2013;131:295-9. e1-27). The step-up options include topical calcineurin inhibitors, phototherapy, and immunosuppressive agents such as oral corticosteroids, cyclosporine, azathioprine, methotrexate, and mycophenolate. Amongst these, cyclosporine is approved for treatment of moderate to severe AD in many European countries, but not in the United States, and its use is limited to patients aged 16 years and over (for a maximum of 8 weeks [NEORAL]). Even in cases where cyclosporine has demonstrated substantial efficacy, approximately 50% of patients relapse within 2 weeks, and 80% relapse within 6 weeks after cessation of therapy (Amor K T, et al., J Am Acad Dermatol 2010;63:925-46). Cyclosporine A (CsA) is a potent immunosuppressant affecting both humoral and cellular immune responses, which could lead to increased susceptibility to infections and decreased cancer immunosurveillance. Other commonly recognized toxicities of CsA include hypertension and impaired renal and hepatic function. In addition, CsA interacts with other commonly used medicines potentially affecting their metabolism and effect.

There remains an unmet medical need for safer and more effective therapies and treatment regimens for moderate to severe AD, especially for patients whose AD are not adequately controlled with cyclosporine or cyclosporine is medically inadvisable for the patient. There is also a need for therapeutic treatments and treatment regimens that provide an improved safety profile with limited toxicity compared to existing treatments or provide more tolerability or convenience for patients, thereby improving patient compliance.

### SUMMARY OF INVENTION

Provided herein are methods, uses and pharmaceutical compositions of anti-IL-13 antibodies, such as lebrikizumab, for treating atopic dermatitis or reducing pruritis associated with atopic dermatitis. Also provided herein are doses and dosing regimens for the methods and uses of anti-IL-13 antibodies, such as lebrikizumab, for treating atopic dermatitis or reducing pruritis associated with atopic dermatitis. In some embodiments, provided herein are methods and uses of anti-IL-13 antibodies for treating atopic dermatitis or reducing pruritis associated with atopic dermatitis in patients with moderate to severe atopic dermatitis that are not adequately controlled with cyclosporine (e.g., inadequate response or intolerance to cyclosporine) or for whom cyclosporine is not medically advisable.

In one aspect, provided herein are methods of treating moderate to severe atopic dermatitis or reducing pruritus associated with atopic dermatitis in a patient in need thereof who had inadequate response or intolerance to cyclosporine, or cyclosporine is medically inadvisable for the patient, which comprise administering to the patient a pharmaceutical composition comprising an anti-IL-13 antibody. In some embodiments, provided herein are methods for treating moderate to severe atopic dermatitis or reducing pruritus, which comprise selecting a patient who has moderate to severe atopic dermatitis and had inadequate response or intolerance to cyclosporine, or cyclosporine is medically inadvisable for the patient, and administering to the patient a pharmaceutical composition comprising an antibody that binds human IL-13. In some embodiments, the patient is aged 12 years and older. In some embodiments, the patient has moderate to severe atopic dermatitis for at least a year. In some embodiments, the patient has an Eczema Area and Severity Index (EASI) score of 16 or greater, an Investigator Global Assessment (IGA) score of 3 or greater, and more than 10% of body surface area (BSA) affected by atopic dermatitis, before administration of the pharmaceutical composition.

Also provided herein are methods for treating moderate to severe atopic dermatitis or reducing pruritus associated with atopic dermatitis, which comprise selecting a patient who (i) is aged 12 years and older; (ii) has chronic atopic dermatitis according to Hanifin and Rajka Criteria for more than a year; (iii) has an EASI score of 16 or greater; (iv) has an IGA score of 3 or greater; (v) has more than 10% of BSA affected by atopic dermatitis; (vi) had inadequate response to topical corticosteroids; and (vii) had inadequate response or intolerance to cyclosporine, or cyclosporine is medically inadvisable for the patient, and administering to the patient a pharmaceutical composition comprising an antibody that binds human IL-13.

In some embodiments, the cyclosporine is cyclosporine A (CsA). In some embodiments, the patient had inadequate response to cyclosporine (e.g., CsA), e.g., at least 4 weeks prior to administering the pharmaceutical composition. In some embodiments, the patient had intolerance to cyclosporine (e.g., CsA). In some embodiments, cyclosporine is medically inadvisable for the patient due to one of the following reasons: (i) medical contraindications, (ii) use of prohibited concomitant medications, (iii) increased susceptibility to cyclosporine-induced renal damage and/or liver damage, (iv) increased risk of serious infections, or (v) hypersensitivity to cyclosporine active substance or excipients. In some embodiments, the patient had inadequate response to topical corticosteroids.

In some embodiments, the anti-IL-13 antibody binds IL-13 with high affinity and blocks signaling through the active IL-4Ralpha/IL-13Ralpha1 heterodimer. In some embodiments, the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6. In some embodiments, the anti-IL-13 antibody comprises a VH comprising SEQ ID NO: 7, and a VL comprising SEQ ID NO: 8. In some embodiments, the anti-IL-13 antibody comprises a heavy chain comprising SEQ ID NO: 9, and a light chain comprising SEQ ID NO: 10. In some embodiments, the anti-IL-13 antibody is lebrikizumab.

In some embodiments, the pharmaceutical composition comprises 250 mg or 500 mg of the anti-IL-13 antibody. In some embodiments, the pharmaceutical composition is administered subcutaneously to the patient.

In some embodiments, the patient is treated with the pharmaceutical composition for a period of about 16-52 weeks. In some embodiments, the patient is treated with the pharmaceutical composition for a treatment period (or an induction period), e.g., about 16 weeks. During this treatment period of 16 weeks, the patient is treated with a loading dose of the pharmaceutical composition comprising 500 mg of the antibody once every two weeks for two doses, and a subsequent dose of the pharmaceutical composition comprising 250 mg of the antibody once every two weeks for seven doses.

After completion of the treatment period or induction period, the patient enters a maintenance period, e.g., up to 36 weeks. In some embodiments, the patient is treated with a maintenance dose of the pharmaceutical composition comprising 250 mg of the antibody once every two weeks during the maintenance period.

In another aspect, provided herein are pharmaceutical composition comprising an anti-IL-13 antibody for use in the treatment of moderate to severe atopic dermatitis or reducing pruritus in a patient who had inadequate response or intolerance to cyclosporine, or cyclosporine is medically inadvisable for the patient. Also provided herein are pharmaceutical composition comprising an anti-IL-13 antibody for use in the treatment of moderate to severe atopic dermatitis or reducing pruritus in a patient who (i) is aged 12 years and older; (ii) has chronic atopic dermatitis according to Hanifin and Rajka Criteria for more than a year; (iii) has an EASI score of 16 or greater; (iv) has an IGA score of 3 or greater; (v) has more than 10% of BSA affected by atopic dermatitis; (vi) had inadequate response to topical corticosteroids; and (vii) had inadequate response or intolerance to cyclosporine, or cyclosporine is medically inadvisable for the patient. In some embodiments, the pharmaceutical composition is for subcutaneous administration to the patient.

In another aspect, provided herein are uses of a pharmaceutical composition comprising an anti-IL-13 antibody in the manufacture of a medicament for the treatment of moderate to severe atopic dermatitis or reducing pruritus in a patient who had inadequate response or intolerance to cyclosporine, or cyclosporine is medically inadvisable for the patient. Also provided herein are uses of a pharmaceutical composition comprising an anti-IL-13 antibody in the manufacture of a medicament for the treatment of moderate to severe atopic dermatitis or reducing pruritus in a patient who (i) is aged 12 years and older; (ii) has chronic atopic dermatitis according to Hanifin and Rajka Criteria for more than a year; (iii) has an EASI score of 16 or greater; (iv) has an IGA score of 3 or greater; (v) has more than 10% of BSA affected by atopic dermatitis; (vi) had inadequate response to topical corticosteroids; and (vii) had inadequate response or intolerance to cyclosporine, or cyclosporine is medically inadvisable for the patient. In some embodiments, the pharmaceutical composition is for subcutaneous administration to the patient.

In some embodiments, the methods, uses, and pharmaceutical compositions described herein further comprise administrating one or more topical corticosteroids to the patient. In some embodiments, the topical corticosteroid is triamcinolone acetonide, hydrocortisone, or a combination of triamcinolone acetonide and hydrocortisone. In some embodiments, the topical corticosteroids are administered concomitantly with the anti-IL-13 antibody.

### BRIEF DESCRIPTION OF THE DRAWINGS

FIG. 1 is a schematic diagram of the Phase 3 study design described in Example 1.

### DETAILED DESCRIPTION

Provided herein are methods, uses and pharmaceutical compositions of anti-IL-13 antibodies for treating atopic dermatitis or reducing pruritis associated with atopic dermatitis. Also provided herein are doses and dosing regimens for the methods and uses of anti-IL-13 antibodies for treating atopic dermatitis or reducing pruritis associated with atopic dermatitis. In some embodiments, provided herein are methods and uses of anti-IL-13 antibodies for treating atopic dermatitis or reducing pruritis associated with atopic dermatitis in patients with moderate to severe atopic dermatitis that are not adequately controlled with cyclosporine (e.g., inadequate response or intolerance to cyclosporine) or for whom cyclosporine is not medically advisable.

In one aspect, provided herein are methods of treating moderate to severe atopic dermatitis or reducing pruritus associated with atopic dermatitis in a patient who had inadequate response or intolerance to cyclosporine, or cyclosporine is medically inadvisable for the patient, which comprise administering to the patient a pharmaceutical composition comprising an anti-IL-13 antibody. In some embodiments, provided herein are methods for treating moderate to severe atopic dermatitis or reducing pruritus, which comprise selecting a patient who has moderate to severe atopic dermatitis and had inadequate response or intolerance to cyclosporine, or cyclosporine is medically inadvisable for the patient, and administering to the patient a pharmaceutical composition comprising an antibody that binds human IL-13. In some embodiments, the patient is aged 12 years and older. In some embodiments, the patient has moderate to severe atopic dermatitis for at least a year. In some embodiments, the patient has an EASI score of 16 or greater, an IGA score of 3 or greater, and more than 10% of BSA affected by atopic dermatitis, before administration of the pharmaceutical composition.

Also provided herein are methods for treating moderate to severe atopic dermatitis or reducing pruritus associated with atopic dermatitis, which comprise selecting a patient who (i) is aged 12 years and older; (ii) has chronic atopic dermatitis according to Hanifin and Rajka Criteria for more than a year; (iii) has an EASI score of 16 or greater; (iv) has an IGA score of 3 or greater; (v) has more than 10% of BSA affected by atopic dermatitis; (vi) had inadequate response to topical corticosteroids; and (vii) had inadequate response or intolerance to cyclosporine, or cyclosporine is medically inadvisable for the patient, and administering to the patient a pharmaceutical composition comprising an antibody that binds human IL-13.

In some embodiments, the cyclosporine is cyclosporine A (CsA). In some embodiments, the patient had inadequate response to cyclosporine (e.g., CsA), e.g., at least 4 weeks prior to administering the pharmaceutical composition. In some embodiments, the patient had intolerance to cyclosporine (e.g., CsA). In some embodiments, cyclosporine is medically inadvisable for the patient due to one of the following reasons: (i) medical contraindications, (ii) use of prohibited concomitant medications, (iii) increased susceptibility to cyclosporine-induced renal damage and/or liver damage, (iv) increased risk of serious infections, or (v) hypersensitivity to cyclosporine active substance or excipients. In some embodiments, the patient had inadequate response to topical corticosteroids.

In some embodiments, the patient has prior exposure to dupilumab, an anti-IL-4Ra monoclonal antibody for treating moderate to severe atopic dermatitis. In some embodiments, the patient has no prior exposure to dupilumab.

In some embodiments, the moderate to severe atopic dermatitis can be determined by Hanifin and Rajka criteria. Hanifin and Rajka diagnostic criteria are described in Acta Derm Venereol (Stockh) 1980; Suppl 92:44-7. To establish a diagnosis of atopic dermatitis, the patient requires the presence of at least three “basic features” and three or more minor features listed below. The basic features include pruritus, typical morphology and distribution such as flexural lichenification or linearity, chronic or chronically-relapsing dermatitis, and personal or family history of atopy, such as asthma, allergic rhinitis, atopic dermatitis. The minor features include xerosis, ichthyosis, palmar hyperlinearity, or keratosis pilaris, immediate (type 1) skin-test reactivity, elevated serum IgE, early age of onset, tendency toward cutaneous infections (especially S. aureus and Herpes simplex), impaired cell-mediated immunity, tendency toward non-specific hand or foot dermatitis, nipple eczema, cheilitis, recurrent conjunctivitis, Dennie-Morgan infraorbital fold, keratoconus, anterior subcapsular cataracts, orbital darkening, facial pallor/facial erythema, pityriasis alba, anterior neck folds, and itch when sweating. Additional minor criteria include intolerance to wool and lipid solvents, perifollicular accentuation, food intolerance, course influenced by environmental or emotional factors, and white dermographism/delayed blanch.

The severity of atopic dermatitis can also be determined by “Rajka and Langeland criteria,” as described in Rajka G and Langeland T, Acta Derm Venereol (Stockh) 1989; 144 (Suppl): 13-4. Three disease severity assessment categories are scored 1 to 3: i) extent of the body area involved, ii) course, e.g., more or less than 3 months during one year or continuous course, and iii) intensity, ranging from mild itch to severe itch, usually disturbing night's sleep. Scores of 1.5 or 2.5 are allowed. Overall disease severity is determined by the sum of individual scores from the three disease assessment categories and the severity is determined by the sum of these scores with mild defined as a total score of 3-4, moderate as score of 4.5-7.5, and severe as a total score of 8-9.

The anti-IL-13 antibodies suitable for use in the methods and uses provided herein have been described previously, e.g., WO2005062967. In some embodiments, the anti-IL-13 antibody binds IL-13 with high affinity and blocks signaling through the active IL-4Ralpha/IL-13Ralpha1 heterodimer. In some embodiments, the anti-IL-13 antibody comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6. In some embodiments, the anti-IL-13 antibody comprises a VH comprising SEQ ID NO: 7, and a VL comprising SEQ ID NO: 8. In some embodiments, the anti-IL-13 antibody comprises a heavy chain comprising SEQ ID NO: 9, and a light chain comprising SEQ ID NO: 10. In some embodiments, the anti-IL-13 antibody is lebrikizumab. The amino acid sequences of lebrikizumab are provided in Table 1. C-terminal clipping of IgG antibodies could occur where one or two C-terminal amino acids are removed from the heavy chain of the IgG antibodies. For example, if a C-terminal lysine (K) is present, it may be truncated or clipped off from the heavy chain. A penultimate glycine (G) may also be truncated or clipped off from the heavy chain as well. Modification of N-terminal amino acid of IgG could also occur. For example, the N-terminal glutamine (Q) or glutamic acid (E) can cyclize into pyro-glutamate (pE) spontaneously. SEQ ID NO: 9 reflects these potential modifications of lebrikizumab heavy chain.

TABLE 1
Lebrikizumab Sequences

| SEQ ID NO: | Description | Sequence |
|---|---|---|
| 1 | Lebrikizumab | AYSVN |
| | HCDR1 | |
| 2 | Lebrikizumab | MIWGDGKIVYNSALKS |
| | HCDR2 | |
| 3 | Lebrikizumab | DGYYPYAMDN |
| | HCDR3 | |
| 4 | Lebrikizumab | RASKSVDSYGNSFMH |
| | LCDR1 | |
| 5 | lebrikizumab | LASNLES |
| | LCDR2 | |
| 6 | Lebrikizumab | QQNNEDPRT |
| | LCDR3 | |
| 7 | Lebrikizumab | VTLRESGPALVKPTQTLTLTCTVSGFSLSAYSVNWIR |
| | heavy chain | QPPGKALEWLAMIWGDGKIVYNSALKSRLTISKDTSK |
| | variable region | NQVVLTMTNMDPVDTATYYCAGDGYYPYAMDNWG |
| | (VH) | QGSLVTVSS |
| 8 | Lebrikizumab | DIVMTQSPDSLSVSLGERATINCRASKSVDSYGNSF |
| | light chain | MHWYQQKPGQPPKLLIYLASNLESGVPDRFSGSGS |
| | variable region | GTDFTLTISSLQAEDVAVYYCQQNNEDPRTFGGGTK |
| | (VL) | VEIK |
| 9 | Lebrikizumab | Xaa1VTLRESGPALVKPTQTLTLTCTVSGFSLSAYSV |
| | heavy chain | NWIRQPPGKALEWLAMIWGDGKIVYNSALKSRLTISK |
| | (HC) | DTSKNQVVLTMTNMDPVDTATYYCAGDGYYPYAMD |
| | | NWGQGSLVTVSSASTKGPSVFPLAPCSRSTSESTAA |
| | | LGCLVKDYFPEPVTVSWNSGALTSGVHTFPAVLQSS |
| | | GLYSLSSVVTVPSSSLGTKTYTCNVDHKPSNTKVDK |
| | | RVESKYGPPCPPCPAPEFLGGPSVFLFPPKPKDTLMI |
| | | SRTPEVTCVVVDVSQEDPEVQFNWYVDGVEVHNAK |
| | | TKPREEQFNSTYRVVSVLTVLHQDWLNGKEYKCKVS |
| | | NKGLPSSIEKTISKAKGQPREPQVYTLPPSQEEMTKN |
| | | QVSLTCLVKGFYPSDIAVEWESNGQPENNYKTTPPV |
| | | LDSDGSFFLYSRLTVDKSRWQEGNVFSCSVMHEAL |
| | | HNHYTQKSLSLSLXaa2Xaa3 |
| | | wherein Xaa1 is Q, pE, or absent; Xaa2 is G or absent; |
| | | Xaa3 is K or absent. |
| 10 | Lebrikizumab | DIVMTQSPDSLSVSLGERATINCRASKSVDSYGNSF |
| | light chain (LC) | MHWYQQKPGQPPKLLIYLASNLESGVPDRFSGSGS |
| | | GTDFTLTISSLQAEDVAVYYCQQNNEDPRTFGGGTK |
| | | VEIKRTVAAPSVFIFPPSDEQLKSGTASVVCLLNNFYP |
| | | REAKVQWKVDNALQSGNSQESVTEQDSKDSTYSLS |
| | | STLTLSKADYEKHKVYACEVTHQGLSSPVTKSFNRG |
| | | EC |

The anti-IL-13 antibodies, e.g., lebrikizumab, can be formulated with suitable carriers or excipients into a pharmaceutical composition that is suitable for administration to patients. For example, the anti-IL-13 antibodies, e.g., lebrikizumab, can be formulated in a pharmaceutical composition as described in WO 2013/066866. The pharmaceutical composition can comprise 100 mg, 150 mg, 200 mg, 250 mg, 300 mg, 350 mg, 400 mg, 450 mg, or 500 mg of the anti-IL-13antibody. In some embodiments, the pharmaceutical composition comprises 250 mg or 500 mg of the anti-IL-13 antibody. In some embodiments, the anti-IL-13 antibody concentration in the pharmaceutical composition is between 100 mg/ml and 150 mg/ml, e.g., 125 mg/mL. The pharmaceutical composition can also comprise 5 mM-40 mM histidine acetate buffer, pH 5.4 to 6.0. In some embodiments, the pharmaceutical composition further comprises a polyol (e.g., sugar) that has a concentration between 100 mM and 200 mM, and/or a surfactant (e.g., polysorbate 20) that has a concentration of 0.01%-0.1%. In one embodiment, the pharmaceutical composition comprises 125 mg/ml of anti-IL-13 antibody (e.g., lebrikizumab), 20 mM histidine acetate buffer, pH 5.7, 175 mM sucrose and 0.03% polysorbate 20.

In some embodiments, the pharmaceutical composition is administered subcutaneously to the patient. The pharmaceutical composition can be administered to the patient at a dosing frequency of about once a week, once every two weeks, once every three weeks, once every four weeks, once every five weeks, once every six weeks, or once every eight weeks. In some embodiments, the pharmaceutical composition is administered to the patient once every two weeks or once every four weeks. In some embodiments, the pharmaceutical composition comprising 250 mg or 500 mg of the anti-IL-13 antibody is administered subcutaneously to the patient once every two weeks or once every four weeks.

In some embodiments, the pharmaceutical composition is administered to the patient using a subcutaneous administration device. The subcutaneous administration device can be selected from a prefilled syringe, disposable pen injection device, microneedle device, microinfuser device, needle-free injection device, or autoinjector device. Various subcutaneous administration devices, including autoinjector devices, are known in the art and are commercially available. Exemplary devices include, but are not limited to, prefilled syringes (such as BD HYPAK SCF®, READYFILL™, and STERIFILL SCF™ from Becton Dickinson; CLEARSHOT™ copolymer prefilled syringes from Baxter; and Daikyo Seiko CRYSTAL ZENITH® prefilled syringes available from West Pharmaceutical Services); disposable pen injection devices such as BD Pen from Becton Dickinson; ultra-sharp and microneedle devices (such as INJECT-EASE™ and microinfuser devices from Becton Dickinson; and H-PATCH™ available from Valeritas) as well as needle-free injection devices (such as BIOJECTOR® and IJECT® available from Bioject; and SOF-SERTER® and patch devices available from Medtronic). In some embodiments, the subcutaneous administration device is an autoinjector device described in WO 2008/112472, WO 2011/109205, WO 2014/062488, and/or WO 2016/089864.

In some embodiments, the patient is treated with the pharmaceutical composition for a period of about 16-52 weeks, e.g., 16 weeks, 20 weeks, 24 weeks, 28 weeks, 32 weeks, 36 weeks, 40 weeks, 44 weeks, 48 weeks, 52 weeks.

In some embodiments, the patient is treated with the pharmaceutical composition for a treatment period (or an induction period) of about 16 weeks. During the treatment period of 16 weeks, the patient is treated with a loading dose comprising 500 mg of the antibody once every two weeks for two doses (e.g., at baseline (Week 0) and Week 2), and a subsequent dose comprising 250 mg of the antibody once every two weeks for seven doses (e.g., at week 4, week 6, week 8, week 10, week 12, week 14 and week 16).

During and after the treatment period, the patient can be assessed for one or more characteristics of the Atopic Dermatitis Disease Severity Measures (ADDSM), which determine certain signs, symptoms, features, or parameters that have been associated with atopic dermatitis and that can be quantitatively or qualitatively assessed. Exemplary ADDSM include, but are not limited to, Eczema Area and Severity Index (EASI), Investigator Global Assessment (IGA), body surface area (BSA), Severity Scoring of Atopic Dermatitis (SCORAD), Pruritus Numerical Rating Scale (NRS), Sleep loss scale, Skin pain NRS score, Patient-Oriented Eczema Measure (POEM) total score, Dermatology Life Quality Index (DLQI) score, Children Dermatology Life Quality Index (CDLQI), DLQI-Relevant (DLQI-R) score, World Health Organization-Five Well-Being Index (WHO-5) score, Recap of Atopic Eczema (RECAP) score, Treatment Satisfaction Questionnaire for Medication-9 items (TSQM-9) score.

The ADDSM can be measured at baseline (prior to the administration of the pharmaceutical composition) and at one or more time points after administration of the pharmaceutical composition. For example, an ADDSM may be measured at the end of week 1, week 2, week 3, week 4, week 5, week 6, week 7, week 8, week 9, week 10, week 11, week 12, week 13, week 14, week 15, week 16, or longer after the initial treatment with a pharmaceutical composition. The difference between the value of the ADDSM at a particular time point following initiation of treatment and the value of the ADDSM at baseline is used to establish whether there has been an improvement (e.g., a reduction) in the ADDSM.

The “Eczema Area and Severity Index” or “EASI” is a validated measure used in clinical settings to assess the severity and extent of AD (Hanifin et al., Exp Dermatol. 2001; 10:11-18). EASI is a composite index with scores ranging from 0 to 72, with the higher values indicating more severe and or extensive disease. The severity of erythema, induration/papulation, excoriation, and lichenification can be assessed by a clinician or other medical professional on a scale of 0 (absent) to 3 (severe) for each of the 4 body areas: head and neck, trunk, upper limbs, and lower limbs, with half points allowed. In addition, the extent of AD involvement in each of the 4 body areas can be assessed as a percentage by body surface area of head, trunk, upper limbs, and lower limbs, and converted to a score of 0 to 6. A total score (0-72) is assigned based on the sum of total scores for each of the four body region scores.

The “Investigator Global Assessment” or “IGA” is an assessment measure used globally to rate the severity of the patient's AD (Simpson E, et al. J Am Acad Dermatol. 2020;83 (3): 839-846). It is based on a 5-point scale ranging from 0 (clear) to 4 (severe) and a score is selected using descriptors that best describe the overall appearance of the lesions at a given time point (see Table 2).

TABLE 2
Investigator Global Assessment

| Score | Grade | Definition |
|---|---|---|
| 0 | Clear | Minor, residual discoloration; no erythema or |
| | | induration/papulation; no oozing/crusting; no edema. |
| 1 | Almost clear | Trace, faint pink erythema with barely perceptible |
| | | induration/papulation and no oozing/crusting; no |
| | | edema. |
| 2 | Mild | Faint-pink erythema with papulation and edema |
| | | perceptible upon palpation and no oozing/crusting; |
| | | minimal induration. |
| 3 | Moderate | Pink-red erythema with definite edema of skin |
| | | papules and plaques; there may be some |
| | | oozing/crusting; palpable induration. |
| 4 | Severe | Deep/bright red erythema with significant swelling |
| | | and obvious raised borders of papules and plaques |
| | | with oozing/crusting; significant induration. |

The body surface area (BSA) assessment estimates the extent of disease or skin involvement with respect to AD and is expressed as a percentage of total body surface. BSA is determined by a clinician or other medical professional using the patient palm is about 1% BSA rule.

The “Severity Scoring of Atopic Dermatitis” or “SCORAD” is a validated clinical tool for assessing the extent and intensity of AD developed by the European Task Force on Atopic Dermatitis (Consensus report of the European Task Force on Atopic Dermatitis. Dermatology. 1993;186 (1): 23-31). There are 3 components to the assessment: (i) the extent of AD is assessed as a percentage of each defined body area and reported as the sum of all areas, with a score ranging from 0 to 100; (ii) the intensity part of the SCORAD consists of 6 items: redness, swelling, oozing/crusting, scratch marks, skin thickening/lichenification, dryness. Each item is graded is graded as follows: none (0), mild (1), moderate (2), or severe (3) (for a maximum of 18 total points); (iii) subjective assessment of itch and of sleeplessness is recorded for each symptom using a visual analogue scale (VAS), where 0 is no itch (or sleeplessness) and 10 is the worst imaginable itch (or sleeplessness; with a maximum possible score of 20). The SCORAD Index formula is: A/5 +7B/2 +C. In this formula A is defined as the extent (0-100), B is defined as the intensity (0-18) and C is defined as the subjective symptoms (0-20). The maximal score of the SCORAD Index is 103.

Pruritus Numerical Rating Scale (NRS) is an 11-point scale used by patients (and if applicable, with help of parents/caregiver if required) to rate their worst itch severity over the past 24 hours with 0 indicating “No itch” and 10 indicating “Worst itch imaginable” (Phan N Q, et al. Acta Derm Venereol 2012; 92:502-507). Assessments are recorded by the patient daily up until Week 16 and weekly from Week 16 onwards using an electronic diary. The baseline pruritus NRS is determined based on the average of daily Pruritus NRS during the 7 days immediately preceding baseline. A minimum of 4 daily scores out of the 7 days immediately preceding baseline is required for this calculation.

The Skin Pain NRS is an 11-point scale completed by patients (and if applicable, with help of parents/caregiver if required) to rate their worst skin pain (for example discomfort or soreness) severity over the past 24 hours with 0 indicating “No pain” and 10 indicating “Worst pain imaginable” (Newton L, et al. J Patient Rep Outcomes. 2019 Jul. 16; 3:42). Assessments are recorded by the patient daily up until Week 16 and weekly from Week 16 onwards using an electronic diary. The baseline Skin Pain NRS is determined based on the average of daily Skin Pain NRS during the 7 days immediately preceding baseline. A minimum of 4 daily scores out of the 7 days immediately preceding baseline is required for this calculation.

Sleep loss scale rates patient's sleep on a 5-point Likert scale for inference with sleep (with scores ranging from 0 [not at all], 1 [a little], 2 [moderately], 3 [quite a bit], to 4 [unable to sleep at all]). It is assessed by patients using a Patient-Reported Outcome (PRO) instrument e.g., eDiary.

The Patient-Oriented Eczema Measure (POEM) is a 7-item, validated, questionnaire completed by the patient (and if applicable, with help of parents/caregiver if required) to assess disease symptoms (Centre of Evidence Based Dermatology. POEM-Patient Oriented Eczema Measure. https://www.nottingham.ac.uk/research/groups/cebd/resources/poem.aspx). Patients are asked to respond to questions on skin dryness, itching, flaking, cracking, sleep loss, bleeding, and weeping. All answers carry equal weight with a total possible score from 0 to 28 (answers scored as: No days=0; 1-2 days =1; 3-4 days =2; 5-6 days =3; everyday =4). A high score is indicative of a poor quality of life. POEM responses are captured weekly using an electronic diary.

The Dermatology Life Quality Index (DLQI) is a 10-item, validated questionnaire completed by the patient or caregiver, used to assess the impact of skin disease on the quality of life of the patient (Finlay, A. Y. and Khan, G. K. 1994. Clinical and Experimental Dermatology 1993 Sep. 23; 19:210-216). The 10 questions cover the following topics: symptoms, embarrassment, shopping and home care, clothes, social and leisure, sport, work or study, close relationships, sex, and treatment, over the previous week. Each question is scored from 0 to 3 (“not at all,” “a little,” “a lot,” and “very much”), giving a total score ranging from 0 to 30. A high score is indicative of a poor quality of life.

For adolescents below the age of 16, the Children DLQI (CDLQI) is employed which is based on a set of 10 questions different from those of the DLQI (Lewis-Jones MS, Finlay AY. British Journal of Dermatology, 1995; 132:942-949).

The DLQI-Relevant (DLQI-R) is a recently developed scoring that adjusts the total score of the DLQI questionnaire for the number of not relevant responses (NRRs) indicated by a patient (Rencz F, et al. Br J Dermatol. 2020;182 (5): 1167-1175).

The World Health Organisation-Five Well-Being Index (WHO-5) assessment is a self-reported measure of current mental wellbeing covering 5 positively worded items, related to positive mood (good spirits, relaxation), vitality (being active and waking up fresh and rested), and general interests (being interested in things) (Topp C W, et al. Psychother Psychosom. 2015;84 (3): 167-176.). Each item is rated on 6-point Likert scale, ranging from 0 (at no time) to 5 (all of the time). The raw scores are transformed to a score from 0 to 100, with lower scores indicating worse well-being.

The Recap of Atopic Eczema (RECAP) is a 7-item patient-reported instrument to capture eczema control, over the previous week (Howells, L., et al. British Journal of Dermatology 2019; 183:524-536). Each item is scored on a 5-point Likert scale, ranging from 0 (very good) to 4 (very bad). A higher score indicates worse experience of eczema control.

The Treatment Satisfaction Questionnaire for Medication-9 items (TSQM-9) is a 9-item measure that assesses the most common dimensions patients use to evaluate their medication (i.e., global satisfaction, effectiveness, and convenience) (Bharmal M, et al. Health Qual Life Outcomes. 2009;7:36). The results for each scale are presented from 0 to 100, where higher scores represent better satisfaction.

In some embodiments, the EASI score of the patient is determined after the treatment period, e.g., at Week 16. In some embodiments, the patient's EASI score determined after the treatment period is reduced by 50% or greater compared to the EASI score determined prior to administration of the first loading dose of the antibody, which means the patient has achieved “EASI 50”. In some embodiments, the patient's EASI score determined after the treatment period is reduced by 75% or greater compared to the EASI score determined prior to administration of the first loading dose of the antibody, which means the patient has achieved “EASI 75”. In some embodiments, the patient's EASI score determined after the treatment period is reduced by 90% or greater compared to the EASI score determined prior to administration of the first loading dose of the antibody, which means the patient has achieved “EASI 90”.

In some embodiments, the IGA score of the patient is determined after the treatment period. In some embodiments, the patient's IGA score determined after the treatment period is 0 or 1 and the IGA score determined after the treatment period is reduced by 2 points or greater compared to the IGA score determined prior to administration of the first loading dose of the antibody.

In some embodiments, the pruritus NRS score of the patient is determined after the treatment period. In some embodiments, the patient's pruritus NRS score determined after the treatment period is reduced by 4 points or greater compared to the pruritus NRS score determined prior to administration of the first loading dose of the antibody.

After completion of the treatment period or induction period, the patient enters a maintenance period. The maintenance period can be up to 36 weeks (e.g., about 4 weeks, 8 weeks, 12 weeks, 16 weeks, 20 weeks, 24 weeks, 28 weeks, 32 weeks, 36 weeks). In some embodiments, the patient is treated with a maintenance dose of the pharmaceutical composition comprising 250 mg of the antibody once every two weeks during the maintenance period.

During and after the maintenance period, the patient is assessed for one or more characteristics of the ADDSM, e.g., EASI, IGA, BSA, SCORAD, Pruritus NRS, Sleep loss scale, Skin pain NRS score, POEM total score, DLQI score, CDLQI, DLQI-R score, WHO-5 score, RECAP score, TSQM-9 score. The ADDSM can be measured at the beginning of the maintenance period and at one or more time points during the maintenance period. For example, an ADDSM may be measured at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, or week 36 of the maintenance period. The difference between the value of the ADDSM at a particular time point during the maintenance period and the value of the ADDSM at the beginning of the maintenance period is used to establish whether there has been an improvement (e.g., a reduction) in the ADDSM.

In some embodiments, the EASI score of the patient is determined during or after the maintenance period. In some embodiments, the patient has achieved EASI 50, EASI 75, or EASI 90 during or after the maintenance period compared to the EASI score determined after the treatment period. In some embodiments, the IGA score of the patient is determined during or after the maintenance period. In some embodiments, the patient's IGA score determined during or after the maintenance period is 0 or 1 and the IGA score determined during or after the maintenance period is reduced by 2 points or greater compared to the IGA score determined after the treatment period. In some embodiments, the pruritus NRS score of the patient is determined during or after the maintenance period. In some embodiments, the patient's pruritus NRS score determined during or after the maintenance period is reduced by 4 points or greater compared to the pruritus NRS score determined after the treatment period.

In another aspect, provided herein are pharmaceutical composition comprising an anti-IL-13 antibody for use in the treatment of moderate to severe atopic dermatitis or reducing pruritus in a patient who had inadequate response or intolerance to cyclosporine, or cyclosporine is medically inadvisable for the patient. Also provided herein are pharmaceutical composition comprising an anti-IL-13 antibody for use in the treatment of moderate to severe atopic dermatitis or reducing pruritus in a patient who (i) is aged 12 years and older; (ii) has chronic atopic dermatitis according to Hanifin and Rajka Criteria for more than a year; (iii) has an EASI score of 16 or greater; (iv) has an IGA score of 3 or greater; (v) has more than 10% of BSA affected by atopic dermatitis; (vi) had inadequate response to topical corticosteroids; and (vii) had inadequate response or intolerance to cyclosporine, or cyclosporine is medically inadvisable for the patient. In some embodiments, the pharmaceutical composition is for subcutaneous administration to the patient.

In another aspect, provided herein are uses of a pharmaceutical composition comprising an anti-IL-13 antibody in the manufacture of a medicament for the treatment of moderate to severe atopic dermatitis or reducing pruritus in a patient who had inadequate response or intolerance to cyclosporine, or cyclosporine is medically inadvisable for the patient. Also provided herein are uses of a pharmaceutical composition comprising an anti-IL-13 antibody in the manufacture of a medicament for the treatment of moderate to severe atopic dermatitis or reducing pruritus in a patient who (i) is aged 12 years and older; (ii) has chronic atopic dermatitis according to Hanifin and Rajka Criteria for more than a year; (iii) has an EASI score of 16 or greater; (iv) has an IGA score of 3 or greater; (v) has more than 10% of BSA affected by atopic dermatitis; (vi) had inadequate response to topical corticosteroids; and (vii) had inadequate response or intolerance to cyclosporine, or cyclosporine is medically inadvisable for the patient. In some embodiments, the pharmaceutical composition is for subcutaneous administration to the patient.

In some embodiments, the methods and uses described herein further comprise administrating one or more topical corticosteroids to the patient. Exemplary topical corticosteroids include, but are not limited to, triamcinolone acetonide, hydrocortisone, and a combination of triamcinolone acetonide and hydrocortisone. Triamcinolone acetonide is typically formulated at a concentration of 0.1% in a cream, and hydrocortisone is typically formulated at a concentration of 1% or 2.5% in a cream. Certain topical corticosteroids are considered very high potency such as, for example, betamethasone dipropionate, clobetasol propionate, diflorasone diacetate, fluocinonide, and halobetasol propionate. Certain topical corticosteroids are considered high potency such as, for example, amcinonide, desoximetasone, halcinonide, and triamcinolone acetonide. Certain topical corticosteroids are considered medium potency, such as, for example, betamethasone valerate, clocortolone pivalate, fluocinolone acetonide, flurandrenolide, fluocinonide, fluticasone propionate, hydrocortisone butyrate, hydrocortisone valerate, mometasone furoate, and prednicarbate. Certain topical corticosteroids are considered low potency, such as, for example, alclometasone dipropionate, desonide, and hydrocortisone. TCS may be applied to affected areas once daily, twice daily, three times per day, or as needed. In some embodiments, the patient is inadequately controlled on topical corticosteroids. In some embodiments, the topical corticosteroid is triamcinolone acetonide, hydrocortisone, or a combination of triamcinolone acetonide and hydrocortisone. In some embodiments, the topical corticosteroids are administered concomitantly or sequentially with the anti-IL-13 antibody. In some embodiments, the topical corticosteroids are administered concomitantly with the anti-IL-13 antibody.

As used herein, the term “a,” “an,” “the” and similar terms used in the context of the present disclosure (especially in the context of the claims) are to be construed to cover both the singular and plural unless otherwise indicated herein or clearly contradicted by the context.

The term “about” as used herein, means in reasonable vicinity of the stated numerical value, such as plus or minus 10% of the stated numerical value.

The term “antibody,” as used herein, refers to an immunoglobulin molecule that binds an antigen. Embodiments of an antibody include a monoclonal antibody, polyclonal antibody, human antibody, humanized antibody, chimeric antibody, or conjugated antibody. The antibodies can be of any class (e.g., IgG, IgE, IgM, IgD, IgA) and any subclass (e.g., IgG1, IgG2, IgG3, IgG4).

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Exemplary embodiments of antibodies of the present disclosure also include antibody fragments or antigen-binding fragments, which comprise at least a portion of an antibody retaining the ability to specifically interact with an antigen such as Fab, Fab′, F(ab′)₂, Fv fragments, scFv, scFab, disulfide-linked Fvs (sdFv), a Fd fragment and linear antibodies.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

The term “flare” as used herein refers to increase in signs and/or symptoms leading to escalation of therapy, which can be an increase in dose, a switch to a higher-potency class of drug, or the start of another drug.

The term “high affinity” as used herein refers to the strength of binding of an antibody to human IL-13 with an equilibrium dissociation constant (K_{D}) of less than about 10⁻⁸ M, e.g., from 10⁻¹⁵ M to 10⁻⁸ M, or from 10⁻¹² M to 10⁻⁹ M.

The term “human IL-13” refers to human interleukin 13 (also known as P600), an immunoregulatory cytokine produced primarily by activated Th2 cells. There are two known human IL-13 isoforms: isoform a and isoform b. The term “human IL-13” as used herein refers collectively to all human IL-13 isoforms. The amino acid sequence for human IL-13 isoform a can be found at NCBI Accession No. NP_002179.2. The amino acid sequence for human IL-13 isoform b can be found at NCBI Accession No. NP_001341922.1.

The term “inadequate response” as used herein refers to inability to achieve good disease control of atopic dermatitis (e.g., not able to achieve IGA ≤2 or EASI-75) after use of the treatment for the duration recommended by the product prescribing information, or flare of atopic dermatitis occurs while on the treatment.

The term “intolerance” or “intolerant” as used herein refers to unacceptable toxicity (e.g., elevated creatinine, elevated liver function tests, uncontrolled hypertension, paranesthesia, headache, nausea, hypertrichosis), or requirement for a drug at doses or duration beyond those specified in the prescribing information.

The term “patient”, as used herein, refers to a human patient.

The term “topical corticosteroid” or “TCS”, as used herein includes Group I, Group II, Group III and Group IV topical corticosteroids. According to the Anatomical Therapeutic Chemical (ATC) Classification System of World Health Organization, the corticosteroids are classified as weak (Group I), moderately potent (Group II) and potent (Group III) and very potent (Group IV), based on their activity as compared to hydrocortisone. Group IV TCS (very potent) are up to 600 times as potent as hydrocortisone and include clobetasol and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent) are 2 to 25 times as potent as hydrocortisone and include, but are not limited to, clobetasone butyrate, and triamcinolone acetonide. Group I TCS (weak or mild) includes hydrocortisone, prednisolone, and methylprednisolone.

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### EXAMPLES

### Example 1. A Randomized, Double-Blind, Placebo-Controlled Phase 3 Clinical Trial to Assess the Efficacy and Safety of Lebrikizumab in Combination with Topical Corticosteroids in Adult and Adolescent Patients with Moderate-To-Severe Atopic Dermatitis That Are Not Adequately Controlled with Cyclosporine or For Whom Cyclosporine is Not Medically Advisable.

This is a randomized, double-blind, placebo-controlled, parallel-group study which is 72 weeks in duration (up to 4 weeks of Screening, 52 weeks of treatment [last dose given at Week 50], and 18 weeks of post-last dose safety follow-up). The study is designed to confirm the efficacy and safety of lebrikizumab administered concomitantly with TCS in adolescents and adults with moderate-to-severe AD not adequately controlled with cyclosporine or for whom cyclosporine is not medically advisable.

The study has two treatment periods: a 16-week double-blind treatment period (or induction period) followed by a 36-week open label maintenance period. The study will be double-blind until Week 18 and open-label from Week 20 onward. The patients who had received placebo during the initial treatment period (or induction period) will receive loading doses of lebrikizumab at Weeks 16 and 18. To maintain blinding at Weeks 16 and 18, all patients will receive 2 injections at Weeks 16 and 18 (either 2 injections of lebrikizumab or 1 injection of lebrikizumab and 1 injection of placebo). From Week 20 onward, all patients will receive 1 injection of lebrikizumab 250 mg Q2W.

### Objectives:

This study is designed to evaluate efficacy and safety of lebrikizumab with concomitant TCS through Week 52 in adults and adolescents (aged ≥12 to <18 years and weighing ≥40 kg) with moderate-to-severe AD, who are not adequately controlled with cyclosporine or for whom cyclosporine is not medically advisable.

The primary objective is to evaluate the efficacy of lebrikizumab compared with placebo in patients not adequately controlled with cyclosporine or for whom cyclosporine is not medically advisable up to Week 16.

The secondary objectives include: (1) to evaluate the efficacy in patients not adequately controlled with cyclosporine or for whom cyclosporine is not medically advisable between Week 16 up to Week 52; (2) to evaluate the safety and tolerability of lebrikizumab in patients not adequately controlled with cyclosporine or for whom cyclosporine is not medically advisable up to Week 16; (3) to evaluate the safety and tolerability of lebrikizumab in patients not adequately controlled with cyclosporine or for whom cyclosporine is not medically advisable up to Week 68.

The exploratory objective is to identify biomarkers associated with clinical improvement that may be predictors of response to treatment and to explore biomarker modifications after treatment.

### Patient Population

A sufficient number of patients are to be screened to randomize approximately 312 patients with moderate-to-severe AD.

Inclusion Criteria: patients eligible for inclusion in this trial must fulfil all of the following criteria:
1. Adults and adolescents (>12 to <18 years of age and weighing ≥40 kg).
2. Chronic AD (according to Hanifin and Rajka Criteria) that has been present for ≥1 year before the screening visit.
3. EASI score ≥16 at the Baseline Visit.
4. IGA score ≥3 (moderate) (scale of 0 [clear] to 4 [severe]) at the baseline visit.
5. ≥10% body surface area (BSA) of AD involvement at the baseline visit.
6. Documented history by a physician of an inadequate response to existing topical medications within 6 months preceding screening, defined as: inability to achieve good disease control (e.g., not able to achieve IGA ≤2) after use of at least a moderate-potency TCS for at least 4 weeks, or for the maximum duration recommended by the product prescribing information (e.g., 14 days for high/very high-potency TCS), whichever is shorter.
7. Documented history by a physician of:
   (a) Either no previous CsA exposure and not currently a candidate for CsA treatment because of
      i. medical contraindications (e.g., uncontrolled hypertension on medication), or
      ii. use of prohibited concomitant medications (e.g., statins, digoxin, macrolide antibiotics, barbiturates, anti-seizure drugs, nonsteroidal anti-inflammatory drugs, diuretics, angiotensin-converting-enzyme inhibitors, St John's Wort), or
      iii. increased susceptibility to CsA-induced renal damage (elevated creatinine) and/or liver damage (elevated function tests), or
      iv. increased risk of serious infections, or
      v. hypersensitivity to CsA active substance or excipients;
   (b) OR previously exposed to CsA, and CsA treatment should not be continued or restarted because of
      i. intolerance and/or unacceptable toxicity (e.g., elevated creatinine, elevated liver function tests, uncontrolled hypertension, paraesthesia, headache, nausea, hypertrichosis), or
      ii. requirement for CsA at doses or duration beyond those specified in the prescribing information or inadequate response.
8. Completed electronic diary (eDiary) entries for pruritus and sleep-loss for a minimum of 4 of 7 days preceding randomization.
9. Willing and able to comply with all clinic visits and study-related procedures and questionnaires.
10. Remain abstinent or use effective contraception during the study and for a minimum of 18 weeks following the last dose of lebrikizumab or placebo.
11. Patient must provide signed informed consent.

Exclusion Criteria: patients fulfilling any of the following criteria are not eligible for inclusion in this trial:
1. Participation in a prior lebrikizumab clinical study.
2. Treatment with IL-4 or IL-13 antagonists biological therapies before the baseline visit. Exception: prior treatment with dupilumab is allowed in a subset of patients. A wash-out of at least 8 weeks prior to the baseline visit is required for this subpopulation.
3. Treatment with topical corticosteroids within 1 week before the baseline visit.
4. Treatment with topical calcineurin inhibitors or phosphodiesterase-4 inhibitors such as crisaborole or cannabinoids within 2 weeks before the baseline visit.
5. Treatment with any of the following agents within 4 weeks prior to the baseline visit:
   a. Immunosuppressive/immunomodulating drugs (e.g., systemic corticosteroids, cyclosporine, mycophenolate-mofetil, interferon-y, JAK inhibitors, azathioprine, methotrexate).
   b. Phototherapy and photochemotherapy (PUVA) for AD.
6. Treatment with the following prior to the baseline visit:
   a. An investigational drug within 8 weeks or within 5 half-lives (if known), whichever is longer.
   b. B Cell-depleting biologics, including but not limited to rituximab, within 6 months.
   c. Other biologics within 16 weeks or 5 half-lives (if known), whichever is longer.
7. Treatment with a live (attenuated) vaccine within 12 weeks of the baseline visit, planned during the study, or 18 weeks after the study treatment is discontinued.
8. History of anaphylaxis as defined by the Sampson criteria.
9. Regular use (more than 2 visits per week) of a tanning booth/parlour within 4 weeks of the screening visit.
10. Uncontrolled chronic disease that might require bursts of oral corticosteroids, e.g., co-morbid severe uncontrolled asthma (defined by an ACQ-5 score ≥1.5 or a history of ≥2 asthma exacerbations within the last 12 months requiring systemic [oral and/or parenteral] corticosteroid treatment or hospitalization for >24 hours).
11. Have had any of the following types of infection within 3 months of screening or develop any of these infections before randomization:
   a. Serious (requiring hospitalization, and/or intravenous or equivalent oral antibiotic treatment);
   b. Opportunistic (as defined in Winthrop et al. 2015). NOTE: Herpes zoster is considered active and ongoing until all vesicles are dry and crusted over.
   c. Chronic (duration of symptoms, signs, and/or treatment of 6 weeks or longer);
   d. Recurring (including, but not limited to herpes simplex, herpes zoster, recurring cellulitis, chronic osteomyelitis).
12. Have a current or chronic infection with hepatitis B virus (HBV).
13. Have a current infection with hepatitis C virus (HCV) (i.e., positive for HCV RNA).
14. Have known liver cirrhosis and/or chronic hepatitis of any etiology.
15. Diagnosed active endoparasitic infections or at high risk of these infections.
16. Known or suspected history of immunosuppression, including history of invasive opportunistic infections (e.g., tuberculosis, histoplasmosis, listeriosis, coccidioidomycosis, pneumocystosis, and aspergillosis) despite infection resolution: or unusually frequent, recurrent, or prolonged infections, per the Investigator's judgment.
17. History of human immunodeficiency virus (HIV) infection or positive HIV serology at screening.
18. In the Investigator's opinion, any clinically significant laboratory results from the chemistry, haematology or urinalysis tests obtained at the screening visit.
19. Presence of skin comorbidities that may interfere with study assessments.
20. History of malignancy, including mycosis fungoides, within 5 years before the screening visit, except completely treated in situ carcinoma of the cervix, completely treated and resolved non-metastatic squamous or basal cell carcinoma of the skin with no evidence of recurrence in the past 12 weeks.
21. Severe concomitant illness(es) that in the Investigator's judgment would adversely affect the patient's participation in the study. Any other medical or psychological condition that in the opinion of the Investigator may suggest a new and/or insufficiently understood disease, may present an unreasonable risk to the study patient because of his/her participation in this clinical trial, may make patient's participation unreliable, or may interfere with study assessments.
22. Pregnant or breastfeeding women, or women planning to become pregnant or breastfeed during the study.
23. Have had an important side effect to TCS (e.g., intolerance to treatment, hypersensitivity reactions, significant skin atrophy, and systemic effects), as assessed by the investigator or treating physician that would prevent further use.

Exclusion criteria includes prior treatment with some medications, which require a washout period before the trial start date (see Table 3).

TABLE 3
Prior Treatment Exclusions and Washout Period

| Drug Class | Wash-out Period |
|---|---|
| IL-4 or IL-13 antagonists | N/A (not allowed)^{a} |
| Topical corticosteroids | 1 week |
| Topical calcineurin inhibitors | 2 weeks |
| Phosphodiesterase-4 inhibitors (e.g., | 2 weeks |
| crisaborole) | |
| Cannabinoids | 2 weeks |
| Immunosuppressive/immunomodulating drugs | 4 weeks |
| (e.g., systemic corticosteroids, cyclosporine, | |
| mycophenolate-mofetil, IFN-γ, JAK inhibitors, | |
| azathioprine, methotrexate) | |
| Phototherapy and photochemotherapy (PUVA) | 4 weeks |
| Investigational drugs | 8 weeks or 5 half-lives |
| | (if known), whichever is |
| | longer |
| B Cell-depleting biologics, including | 6 months |
| but not limited to rituximab | |
| Other biologics | 16 weeks or 5 half-lives |
| | (if known), whichever is |
| | longer |
| Live (attenuated) vaccine | 12 weeks |

Abbreviations: IL= interleukin; IFN = interferon; PUVA = psoralen and ultraviolet A; JAK = Janus kinase.
^{a}Prior treatment with dupilumab is allowed in a subset of patients. A wash-out of at least 8 weeks before the baseline visit is required for this subpopulation.

### Study Drug:

Pharmaceutical compositions containing 125 mg/mL lebrikizumab or placebo are supplied as sterile pre-filled syringes with a pre-assembled needle safety device (PFS-NSD) for subcutaneous administration to the patients. Lebrikizumab sequences are provided in Table 1. The placebo solution is identical in appearance and volume to the active solution except that it does not contain lebrikizumab.

### Study Design:

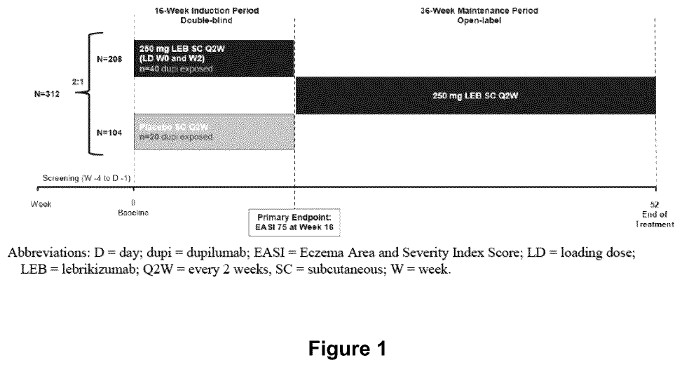

The study has two treatment periods (see FIG. 1): a 16-week double-blind initial treatment period (or Induction Period) followed by a 36-week open label Maintenance Period. The study will be double-blind until Week 18 and open-label from Week 20 onward.

During the 16-week Induction Period, approximately 312 patients are randomized 2:1 to either 250 mg lebrikizumab (loading dose of 500 mg given at Baseline (Week 0) and Week 2) or placebo by SC injection Q2W (once every two weeks). Randomization is stratified by prior use of dupilumab, age (adolescent patients aged ≥12 to <18 years make up to 12.5% of the overall population compared to adults aged ≥18 years) and baseline disease severity (IGA 3 versus 4).

All enrolled patients are required to start treatment with medium-or low-potency TCS at Baseline and continue it throughout the study. A moderate potency TCS, e.g., triamcinolone acetonide 0.1% cream, and a mild TCS, e.g., hydrocortisone 1% cream (for use on sensitive skin areas), are provided for use concomitantly with lebrikizumab in this clinical trial.

After completion of the 16-week Induction Period, patients enter the Maintenance Period. Patients who received lebrikizumab 250 mg Q2W in the Induction Period continue to receive lebrikizumab 250 mg Q2W during the Maintenance Period.

Patients who received placebo in the Induction Period receive loading doses of lebrikizumab as follows: 500 mg lebrikizumab at Weeks 16 and 18. To allow patients in the placebo arm to receive these lebrikizumab loading doses during the Maintenance Period, the blinding will be maintained at Week 16 and Week 18. Therefore, the study is open-label from Week 20 onward. Patients who received placebo in the Induction Period receive lebrikizumab 250 mg Q2W from Week 20 onward during the Maintenance Period.

Response is re-assessed along the Maintenance Period. Patients who do not achieve EASI 50 for at least 2 consecutive visits assessed at Weeks 24, 28, 32, 36, 40, 44, or 48 are discontinued from the study.

### Endpoints

The main objective of this study is to evaluate the efficacy of lebrikizumab compared with placebo in patients not adequately controlled with cyclosporine or for whom cyclosporine is not medically advisable up to Week 16. Efficacy is measured using one or more of the following criteria: (i) Clinical signs: EASI, IGA, BSA affected by AD lesions; (ii) Clinical signs and Patient Reported Symptoms: SCORAD; (iii) AD Patient Reported Symptoms: Pruritus NRS, Sleep-loss scale, Skin Pain NRS, POEM; (iv) Quality of Life (QoL) and impact of disease: DLQI or CDLQI, DLQI-R, WHO-5, RECAP, and TSQM-9.

The primary endpoint for the study is percentage of patients achieving EASI 75 (≥75% reduction from Baseline in EASI score) at Week 16. The secondary endpoints include percentage of patients achieving EASI 90 at Week 16; percentage of patients achieving IGA 0/1 and 2 point improvement at Week 16; percentage of patients achieving a 4 point improvement of Pruritus NRS at Week 16; percentage of patients achieving EASI 90 at Week 16; percentage of patients achieving EASI 75, EASI 90 and EASI 50 (by visit up to Week 16); change from baseline BSA by visit up to Week 16; change from baseline SCORAD by visit up to Week 16; change from baseline Pruritus NRS by visit up to Week 16; change from baseline sleep loss by visit up to Week 16; change from baseline POEM by visit up to Week 16; change from baseline DLQI/CDLQI by visit up to Week 16; percentage of patients achieving a 4 point improvement of DLQI/CDLQI by visit up to Week 16; proportion of TCS-free days from Baseline by visit up to Week 16; time to TCS-free use (days) up to Week 16; change from baseline Skin Pain NRS by visit up to Week 16; percentage of patients achieving a 4-point improvement Skin Pain NRS at Week 16. Other exploratory endpoints include change from baseline RECAP by visit up to Week 16; change from baseline WHO-5 by visit up to Week 16; TSQM-9 by visit up to Week 16; change from baseline DLQI-R by visit up to Week 16; time to EASI 50, EASI 75 and EASI 90 (days) up to Week 16.

The secondary objective is to evaluate the efficacy in patients not adequately controlled with cyclosporine or for whom cyclosporine is not medically advisable between Week 16 up to Week 52. The endpoints for this secondary objective include percentage of patients achieving EASI-75 at Week 16 who continue to exhibit EASI-75 at Week 52; percentage of patients achieving EASI 75, EASI 90, EASI 50 (by visit, between Week 16 and Week 52); percentage of patients achieving IGA 0/1 and 2 point improvement (by visit, between Week 16 and Week 52); percentage of patients achieving a 4 point improvement Pruritus NRS (by visit, between Week 16 and Week 52); change from baseline BSA (by visit, between Week 16 and Week 52); change from baseline SCORAD (by visit, between Week 16 and Week 52); change from baseline Pruritus NRS (by visit, between Week 16 and Week 52); change from baseline sleep loss (by visit, between Week 16 and Week 52); change from baseline POEM (by visit, between Week 16 and Week 52); change from baseline DLQI/CDLQI (by visit, between Week 16 and Week 52); percentage of patients achieving a 4 point improvement in DLQI/CDLQI (by visit, between Week 16 and Week 52); proportion of TCS-free days from Baseline (by visit, between Week 16 and Week 52); time to TCS-free use (days); change from baseline Skin Pain NRS (by visit, between Week 16 and Week 52); percentage of patients achieving a 4 point improvement in Skin Pain NRS (by visit, between Week 16 and Week 52). Other exploratory endpoints include change from baseline RECAP (by visit, between Week 16 and Week 52); change from baseline WHO-5 (by visit, between Week 16 and Week 52); TSQM-9 (by visit, between Week 16 and Week 52); change from baseline DLQI-R (by visit, between Week 16 and Week 52); time to, EASI 75 and EASI 90 (days) (by visit, between Week 16 and Week 52).

To evaluate the safety and tolerability of lebrikizumab in patients not adequately controlled with cyclosporine or for whom cyclosporine is not medically advisable, the incidence of adverse events (AEs) is monitored and assessed, including treatment-emergent adverse event (TEAEs), serious adverse events (SAEs), Related TEAEs, Related SAEs, TEAEs leading to study treatment discontinuation, adverse events of special interest (AESIs) (e.g., conjunctivitis or herpes infection or zoster), and deaths. Blood and urine samples are collected from each patient and subject to laboratory testing such as blood chemistry, haematology, serology, coagulation, and urinalysis testing; the results and changes from Baseline are recorded and assessed. A complete physical examination, which includes at least assessments of the cardiovascular, respiratory, gastrointestinal and neurological systems, is performed, and any abnormalities in physical examination are recorded and assessed. Vital Signs such as systolic and diastolic blood pressure (mmHg), heart rate (beats per minute), respiratory rate (breaths per minute), and body temperature (° C.) are measured and the results and changes from Baseline are recorded and assessed.

An exploratory objective is to identify biomarkers associated with clinical improvement that may be predictors of response to treatment and to explore biomarker modifications after treatment. Blood samples from patients are collected, and transcriptomic, genomic, and protein analysis are performed. Additional association with EASI outcomes is explored.

Statistical analyses are performed for the primary and secondary endpoints.

1. A method of treating moderate to severe atopic dermatitis or reducing pruritus associated with atopic dermatitis in a patient in need thereof, the method comprising administering to the patient a pharmaceutical composition comprising an antibody that binds human IL-13,
wherein the patient had inadequate response or intolerance to cyclosporine, or cyclosporine is medically inadvisable for the patient, and
wherein the antibody comprises a heavy chain variable region (VH) and a light chain variable region (VL), wherein the VH comprises a HCDRI comprising SEQ ID NO: 1, a HCDR2 comprising SEQ ID NO: 2, and a HCDR3 comprising SEQ ID NO: 3, and the VL comprises a LCDRI comprising SEQ ID NO: 4, a LCDR2 comprising SEQ ID NO: 5, and a LCDR3 comprising SEQ ID NO: 6.

2. A method for treating moderate to severe atopic dermatitis or reducing pruritus associated with atopic dermatitis, the method comprising:
selecting a patient who has moderate to severe atopic dermatitis and had inadequate response or intolerance to cyclosporine, or cyclosporine is medically inadvisable for the patient, and
administering to the patient a pharmaceutical composition comprising an antibody that binds human IL-13,
wherein the antibody comprises a heavy chain variable region (VH) and a light chain variable region (VL), wherein the VH comprises a HCDRI comprising SEQ ID NO: 1, a HCDR2 comprising SEQ ID NO: 2, and a HCDR3 comprising SEQ ID NO: 3, and the VL comprises a LCDRI comprising SEQ ID NO: 4, a LCDR2 comprising SEQ ID NO: 5, and a LCDR3 comprising SEQ ID NO: 6.

3. The method of claim 2, wherein the patient has moderate to severe atopic dermatitis for at least a year.

4. The method of claim 3, wherein the moderate to severe atopic dermatitis is determined by Hanifin and Rajka criteria.

5. (canceled)

6. The method of claim 2, wherein the patient has an Eczema Area and Severity Index (EASI) score of 16 or greater, an Investigator Global Assessment (IGA) score of 3 or greater, and more than 10% of body surface area (BSA) affected by atopic dermatitis, before administration of the pharmaceutical composition.

7. The method of claim 2, wherein the patient had inadequate response to topical corticosteroids.

8. The method of claim 2, wherein the patient is aged 12 years and older.

9. A method for treating moderate to severe atopic dermatitis or reducing pruritus associated with atopic dermatitis, the method comprising:
selecting a patient who:
i. is aged 12 years and older;
ii. has chronic atopic dermatitis according to Hanifin and Rajka Criteria for more than a year:
iii. has an EASI score of 16 or greater:
iv. has an IGA score of 3 or greater:
v. has more than 10% of body surface area affected by atopic dermatitis:
vi. had inadequate response to topical corticosteroids; and
vii. had inadequate response or intolerance to cyclosporine, or cyclosporine is medically inadvisable for the patient, and
administering to the patient a pharmaceutical composition comprising an antibody that binds human IL-13,
wherein the antibody comprises a heavy chain variable region (VH) and a light chain variable region (VL), wherein the VH comprises a HCDRI comprising SEQ ID NO: 1, a HCDR2 comprising SEQ ID NO: 2, and a HCDR3 comprising SEQ ID NO: 3, and the VL comprises a LCDRI comprising SEQ ID NO: 4, a LCDR2 comprising SEQ ID NO: 5, and a LCDR3 comprising SEQ ID NO: 6.

10. The method of claim 2, wherein the cyclosporine is cyclosporine A.

11. The method of claim 2, wherein the patient had inadequate response to cyclosporine.

12. The method of claim 11, wherein the patient had inadequate response to cyclosporine at least 4 weeks prior to administering the pharmaceutical composition.

13. The method of claim 2, wherein the patient had intolerance to cyclosporine.

14. The method of claim 2, wherein cyclosporine is medically inadvisable for the patient due to one of the following reasons:
i. medical contraindications,
ii. use of prohibited concomitant medications,
iii. increased susceptibility to cyclosporine-induced renal damage and/or liver damage,
iv. increased risk of serious infections, or
v. hypersensitivity to cyclosporine active substance or excipients.

15. The method of claim 2, wherein the patient had inadequate response to topical corticosteroids at least two weeks prior to administering the pharmaceutical composition.

16. The method of claim 2, wherein the patient has no prior exposure to dupilumab.

17. The method of claim 2, wherein the patient has prior exposure to dupilumab.

18. The method of claim 2, wherein the antibody comprises a VH comprising SEQ ID NO: 7, and a VL comprising SEQ ID NO: 8.

19. The method of claim 2, wherein the antibody comprises a heavy chain comprising SEQ ID NO: 9, and a light chain comprising SEQ ID NO: 10.

20. The method of claim 2, wherein the antibody is lebrikizumab.

21. The method of claim 2, wherein the pharmaceutical composition comprises 250 mg or 500 mg of the antibody.

22. The method of claim 2, wherein the pharmaceutical composition is administered subcutaneously to the patient.

23. The method of claim 2, wherein the pharmaceutical composition is administered subcutaneously to the patient once every two weeks.

24. The method of claim 2, wherein the patient is treated with the pharmaceutical composition for a period of 16-52 weeks.

25. The method of claim 2, wherein the patient is treated with the pharmaceutical composition for a treatment period of 16 weeks.

26. The method of claim 25, wherein, during the treatment period, the patient is treated with a loading dose of the pharmaceutical composition comprising 500 mg of the antibody once every two weeks for two doses, and a subsequent dose of the pharmaceutical composition comprising 250 mg of the antibody once every two weeks for seven doses.

27. The method of claim 25, further comprising determining the EASI score of the patient after the treatment period.

28. The method of claim 27, wherein the EASI score determined after the treatment period is reduced by 50% or greater compared to the EASI score determined prior to administration of the first loading dose of the antibody.

29. The method of claim 27, wherein the EASI score determined after the treatment period is reduced by 75% or greater compared to the EASI score determined prior to administration of the first loading dose of the antibody.

30. The method of claim 27, wherein the EASI score determined after the treatment period is reduced by 90% or greater compared to the EASI score determined prior to administration of the first loading dose of the antibody.

31. The method of claim 25, further comprising determining the IGA score of the patient after the treatment period.

32. The method of claim 31, wherein the IGA score determined after the treatment period is 0 or 1 and the IGA score determined after the treatment period is reduced by 2 points or greater compared to the IGA score determined prior to administration of the first loading dose of the antibody.

33. The method of claim 25, further comprising determining the percentage of BSA affected by atopic dermatitis of the patient after the treatment period.

34. The method of claim 25, further comprising determining the pruritus numeric rating scale (NRS) score of the patient after the treatment period.

35. The method of claim 34, wherein the pruritus NRS score determined after the treatment period is reduced by 4 points or greater compared to the pruritus NRS score determined prior to administration of the first loading dose of the antibody.

36. The method of claim 25, further comprising determining one or more of the following characteristics of the patient after the treatment period:
i. Severity Scoring of Atopic Dermatitis (SCORAD);
ii. Sleep loss scale;
iii. Skin pain NRS score;
iv. Patient-Oriented Eczema Measure (POEM) total score;
V. Dermatology Life Quality Index (DLQI) score, Children Dermatology Life Quality Index (CDLQI), or DLQI-Relevant (DLQI-R) score;
vi. World Health Organisation-Five Well-Being Index (WHO-5) score;
vii. Recap of Atopic Eczema (RECAP) score;
viii. Treatment Satisfaction Questionnaire for Medication-9 items (TSQM-9) score.

37. The method of claim 25, wherein the patient is further treated with the pharmaceutical composition for a maintenance period up to 36 weeks.

38. The method of claim 37, wherein the patient is treated with a maintenance dose of the pharmaceutical composition comprising 250 mg of the antibody once every two weeks during the maintenance period.

39. The method of claim 37, further comprising determining the EASI score of the patient during or after the maintenance period.

40. The method of claim 39, wherein the EASI score determined during or after the maintenance period is reduced by 50% or greater compared to the EASI score determined after the treatment period.

41. The method of claim 39, wherein the EASI score determined during or after the maintenance period is reduced by 75% or greater compared to the EASI score determined after the treatment period.

42. The method of claim 39, wherein the EASI score determined during or after the maintenance period is reduced by 90% or greater compared to the EASI score determined after the treatment period.

43. The method of claim 37, further comprising determining the IGA score of the patient during or after the maintenance period.

44. The method of claim 43, wherein the IGA score determined during or after the maintenance period is 0 or 1 and the IGA score determined during or after the maintenance period is reduced by 2 points or greater compared to the IGA score determined after the treatment period.

45. The method of claim 37, further comprising determining the percentage of BSA affected by atopic dermatitis of the patient during or after the maintenance period.

46. The method of claim 37, further comprising determining the pruritus NRS score of the patient during or after the maintenance period.

47. The method of claim 46, wherein the pruritus NRS score determined during or after the maintenance period is reduced by 4 points or greater compared to the pruritus NRS score determined after the treatment period.

48. The method of claim 37, further comprising determining one or more of the following characteristics of the patient after the maintenance period:
i. SCORAD;
ii. Sleep loss scale;
iii. Skin pain NRS score;
iv. POEM total score;
V. DLQI score, CDLQI, or DLQI-R score;
vi. WHO-5 score;
vii. RECAP score;
viii. TSQM-9 score.

49. The method of claim 2, wherein the pharmaceutical composition is administered to the patient using a subcutaneous administration device.

50. The method of claim 49, wherein the subcutaneous administration device is selected from a prefilled syringe, disposable pen injection device, microneedle device, microinfuser device, needle-free injection device, or autoinjector device.

51. The method of claim 2, wherein the method further comprises administrating one or more topical corticosteroids to the patient.

52. The method of claim 51, wherein the one or more topical corticosteroids is triamcinolone acetonide, hydrocortisone, or a combination of triamcinolone acetonide and hydrocortisone.

53. The method of claim 51, wherein the one or more topical corticosteroids is administered concomitantly with the antibody.

54-59. (canceled)

60. The method of claim 37, wherein the patient is treated with a maintenance dose of the pharmaceutical composition comprising 250 mg of the antibody once every four weeks during the maintenance period.
